(19) United States
(12) Patent Application Publication
(10) Pub. No.: US 2007/0128130 A1
(43) Pub. Date: Jun. 7, 2007

(54) Remineralizing dental adhesive film

(76) Inventors: Christian Kropf, Hilden (DE); Peter Wuelknitz, Leichlingen (DE)

Correspondence Address: PAUL & PAUL, 2000 MARKET STREET, PHILADELPHIA, PA, 19103-3229, US

(21) Appl. No.: 11/649,637

(22) Filed: Jan. 4, 2007

Related U.S. Application Data
(63) Continuation of application No. 10/465,157, filed on Jun. 19, 2003.
Application No. 10/465,157 is a continuation of application No. PCT/EP01/14512, filed on Dec. 11, 2001.

(30) Foreign Application Priority Data
Dec. 20, 2000 (DE) 100 63 945.3

(51) Int. Cl. A61K 8/96; A61K 8/21

(52) U.S. Cl. 424/50; 424/52

(57) ABSTRACT

A dental adhesive film that, when applied to dental material, assists in the remineralization of dental material that exhibits damage from caries, lesions in the enamel and open dentine channels. The active compound in the dental adhesive film is a finely divided, poorly soluble calcium salt of phosphates, fluorides, fluorophosphates and mixtures thereof. Optionally present may be hydroxyl, carbonate or chloride ions.

### CROSS-REFERENCE TO RELATED APPLICATIONS

This application is a continuation of U.S. application Ser. No. 10/465,157, filed on Jun. 19, 2003 which is a continuation under 35 U.S.C. § 365(c) and 35 U.S.C. § 120 of International Application PCT/EP01/14512, filed on Dec. 11, 2001, the International Application not being published in English. This application also claims priority under 35 U.S.C. § 119 to German Application DE 100 63 945.3, filed on Dec. 20, 2000. The entire contents of each of the foregoing applications is incorporated herein by reference.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The invention relates to an adhesive film, which has a certain adhesion to the surface of the tooth or to the gums and is soluble or swellable in water and in which a finely divided, poorly water-soluble calcium salt is incorporated as a remineralizing active compound.

Not only cleansers such as, for example, toothpastes or mouthwashes are used for the care and preservation of the health of the teeth. Lozenges or chewing gum preparations which have a relatively long residence time in the mouth are also suitable for introducing certain active compounds onto the gums or onto the tooth surface. Finally, it has also already been proposed to equip adhesive films which adhere to the gums or to the tooth surface with active compounds against caries or peridontitis.

2. Description of Related Art, Including Information Disclosed Under 37 C.F.R. §§ 1.97 and 1.98.

As one of the first stages of dental caries, lesions in the enamel and open dentine channels (“Tomes pits”) are observed, which result due to dissolving processes under the influence of acid-forming bacteria. The opening of the dentine channels makes itself noticeable, for example, by dental neck sensitivity to temperature variations. While only the painful symptoms are controlled by additions of desensitizing active compounds, attempts have already been made to prevent the formation of such tooth surface lesions by additions which reduce apatite solubility. Recently, proposals have also already been made to reduce existing damage by means of remineralizing toothcare compositions. Thus, it was proposed by Chow and Brown (in J. Dent. Res. 54, (1975), 65-70) to employ dicalcium phosphate dihydrate for the remineralization of the dentine. U.S. Pat. No. 4,097,588 disclosed a mouthwash having remineralizing action, which was saturated with Ca₂HPO₄. 2H₂O.

In European Application No. EP 0 165 454 B1, hydroxyapatite or fluoroapatite in finely divided form (below 4 micrometers particle diameter) is proposed as a component of toothcare compositions.

European Application No. EP 0 381 193 A2 discloses films for application to the oral mucous membrane, which can contain a topical active compound, e.g., also sodium fluoride or potassium nitrate.

Published International Application WO 95/33441 A1 describes phosphate-free compositions which contain finely divided (colloidal) metal compounds, e.g., of yttrium, cerium, aluminum or zirconium for the treatment of hypersensitive teeth and which are also intended to be applied in the form of oral adhesive patches.

The object was, therefore, to find an effective application form for the calcium salts having remineralizing action, in particular, the phosphates, fluorides, fluorophosphates, and also hydroxyapatite and fluoroapatite, which bring about local remineralization of the damaged enamel.

### BRIEF SUMMARY OF THE INVENTION.

This object was achieved according to the invention by a dental adhesive film for local, remineralizing tooth treatment comprising a water soluble or swellable support material for adhering to the tooth, comprising a composite comprising at least one active remineralizing compound incorporated into the support material, wherein the active compound is a poorly water-soluble calcium salt of a compound selected from the group consisting of fluorides, fluorophosphates and mixtures thereof in the form of a finely divided rod-shaped nanoparticle having a mean particle fineness of from 10 to 300 nm and a protein component selected from the group consisting of proteins, and protein degradation products and derivatives of proteins or protein degradation products, wherein the composite is an aggregate which is microscopically heterogeneous but macroscopically homogeneous.

### BRIEF DESCRIPTION OF THE SEVERAL VIEWS OF THE DRAWING(S)

Not Applicable

### DETAILED DESCRIPTION OF THE INVENTION.

The support film can, in this case, consist of any desired solid, flexible material which is soluble or swellable in water. Suitable materials are preferably natural or synthetic polymers which are softened with water and/or water-miscible solvents. An example of such a material is, for example, according to U.S. Pat. No. 3,444,858, a gelatine softened by water and glycerol. Further examples of suitable support materials are, according to Published International Application WO 00/18365 A1, for example, pullulan, hydroxypropylcellulose, hydroxyethylcellulose, hydroxy-propylmethylcellulose, carboxymethylcellulose, sodium alginate, xanthan gum, tragacanth, guar, acacia gum, gum arabic, amylose, hydroxypropyl starch, dextrin, pectin, chitin, chitosan, levan, collagen, zein, gluten, soybean protein, casein, polyvinylpyrrolidone, polyvinyl alcohol, polyethylene glycol, polyacrylic acid, methyl methacrylate/acrylic acid copolymer and mixtures thereof. In a preferred embodiment of the invention, the support component contained is a water-soluble or water-swellable natural or synthetic polymer material selected from vegetable and microbial gums, gelatine, cellulose ethers, copolymers of acrylic or methacrylic acid and esters of acrylic or methacrylic acid, polyvinyl alcohol, partially hydrolyzed polyvinyl acetate, polyvinylpyrrolidone and mixtures thereof.

In the composition of the support material, what especially matters is that the active compounds are released from the support in a controlled manner over a relatively long period, so that the support material does not decompose too rapidly or dissolve too rapidly in the mouth under the action of the saliva, and the active compound is swallowed before it is has begun to act on the tooth or gums.

The disintegration or dissolution of the support material can be delayed by various measures. The release of the active compounds is thus controlled specifically. Such measures are, for example, the crosslinking of the water-soluble polymers, the addition of less water-soluble polymers, the addition of hydrophobic components, e.g., magnesium stearate, or, as proposed in Published International Application WO 99/04764 A1, the use of proteins or cellulose ethers crosslinked with tannic acids or tannin.

The preparation of support films from a suitable support material is carried out according to known processes by preparing a solution of the polymer or of the polymer mixture, dissolving or dispersing the active compounds therein and drying this solution or dispersion in a thin layer on a nonadhering substrate, e.g., a substrate coated with silicone. After the evaporation of the solvent, the finished film can be detached from the substrate and optionally cut into a size suitable for application to the teeth.

Poorly water-soluble calcium salt should be understood as meaning salts which are soluble to less than 0.1% by weight (1 g/l) in water at 20° C. Suitable salts of this type are, for example, calcium hydroxyphosphate (Ca₅[OH(PO₄)₃]) or hydroxyapatite, calcium fluoro-phosphate (Ca₅[F(PO₄)₃]) or fluoroapatite, fluorine-doped hydroxyapatite of the composition Ca₅(PO₄)₃(OH,F) and calcium fluoride (CaF₂) or fluorite or fluorspar, and other calcium phosphates such as di-, tri- or tetracalcium phosphate (Ca₂P₂O₇, Ca₃(PO₄)₂, Ca₄P₂O₉, oxyapatite (Ca₁₀(PO₄)₆O) or nonstoichiometric hydroxy-apatite(Ca_{5-1/2(x+y})(PO₄)₃₋ₓ(HPO₄)ₓ(OH)_{1-y}).

A suitable remineralizing active compound is preferably a finely divided, poorly water-soluble calcium salt which is selected from the group consisting of hydroxyapatite, fluoroapatite and mixtures thereof, since the tooth material, whose restoration is the aim of the remineralization, consists to approximately 95% of hydroxyapatite.

Those only slightly water-soluble calcium salts have proven particularly advantageous which have a mean particle fineness of 10-300 nm (nanometers). The particle fineness should be understood here as meaning the diameter of the particles in the direction of their greatest longitudinal extent. The mean particle fineness relates to a volume-averaged value. Such calcium salts can be prepared, for example, according to the process known from German Application No. DE 198 58 662 A1 in the form of rod-shaped primary particles having thicknesses of 5-50 nm and lengths of 10-150 nm.

In the biological formation process of enamel and of the supportive tissue of the bone, hydroxyapatite is deposited in an ordered manner onto the protein matrix in the tooth or bone, which mainly consists of collagen. The formation of the hard and loadable mineral structure is controlled here by “matrix proteins,” which are formed from collagen and further proteins which deposit on the collagen and thus bring about a controlled mineralization process, “bio-mineralization.”

Proteins also serve as protective colloids which are adsorbed onto the surface of the nanoparticles and prevent these from coagulation and agglomeration and slow crystal growth. Even in the remineralization of the damaged tartar, what matters is that no uncontrolled crystal growth takes place which could form only a loose crystal structure. On the contrary, the crystal growth should be retarded and proceed in a controlled manner as a result of proteins as protective colloid in order that a tight and adequately solid crystal structure can be formed.

In a preferred embodiment, the dental adhesive film according to the invention furthermore contains a protein component, selected from proteins, protein degradation products and derivatives of proteins or protein degradation products.

Suitable proteins here are all proteins independently of their origin, that is both animal and plant proteins. Suitable animal proteins are, for example, collagen, fibroin, elastin, keratin, albumin and casein. Suitable plant proteins are, for example, wheat and wheatgerm proteins (gluten), rice protein, soybean protein, oat protein, pea protein, almond protein and potato protein. Single-cell proteins such as, for example, yeast protein or bacterial proteins are also suitable.

Proteins preferred according to the invention are animal products such as collagen, keratin and casein.

According to a further preferred embodiment, the protein can also originate from a plant or marine source.

Protein degradation products are understood as meaning those products which are obtainable by hydrolytic, oxidative or reductive degradation of water-insoluble proteins to give oligo- and polypeptide structures having relatively low molecular weight and having improved water solubility.

The hydrolytic degradation of water-insoluble proteins is the most important degradation method; it can be carried out under the catalytic influence of acids, alkalis or of enzymes. Protein degradation products preferably suitable are especially those which are not degraded further than necessary for the attainment of the water solubility.

The only slightly degraded protein hydrolyzates include, for example, the gelatines preferred in the context of the present invention, which can have molar masses in the range from 15,000 to 250,000 D. Gelatine is a polypeptide which is obtained mainly by hydrolysis of collagen under acidic (gelatine type A) or alkaline (gelatine type B) conditions. The gel strength of the gelatine is proportional to its molecular weight, i.e. a more strongly hydrolyzed gelatine affords a less viscous solution. The gel strength of the gelatine is indicated in Bloom numbers. In the enzymatic cleavage of gelatine, the polymer size is greatly lowered, which leads to very low Bloom numbers.

Derivatives of proteins and protein degradation products are understood as meaning chemically modified proteins or protein hydrolyzates, which are obtainable, for example, by acylation of free amino groups, by addition of ethylene oxide or propylene oxide and hydroxyl, amino or carboxyl groups or by alkylation of hydroxyl groups of the protein or protein degradation product or of a hydroxyalkyl derivative thereof, e.g., with epoxypropyltrimethylammonium chloride or 3-chloro-2-hydroxypropyltrimethylammonium chloride.

In a particularly preferred embodiment, the protein component is selected from gelatine, casein, their hydrolyzates and mixtures thereof. The dental adhesive film according to the invention can, for example, consist mainly of a protein component, e.g., of gelatine or collagen, as a support material. If, however, the support material used is another material, e.g., a plant gum, a single-cell biopolymer (xanthan gum, pullulan), a cellulose or starch ether, a polyvinylpyrrolidone or a mixture of cellulose ether, polyvinyl acetate and polyacrylic acid, a protein component should preferably be contained therein in an amount of at least 1% by weight, preferably of 1-20% by weight.

A further particularly preferred embodiment consists in the active compound contained being a composite material of the poorly water-soluble calcium salt and a protein component selected from proteins, protein degradation products and derivatives of proteins or protein degradation products. Composite materials are understood here as meaning compound substances which comprise the poorly soluble calcium salts and the protein components and are aggregates which appear microscopically heterogeneous, but macroscopically homogeneous, in which the primary particles of the calcium salts are present on the structure of the protein component in associated form. The proportion of the protein component in such composite materials is between 0.1 and 60% by weight, but preferably between 1.0 and 20% by weight, based on the weight of the composite material.

The preparation of composite materials from hydroxy-apatite and collagen is described, for example, by R. Z. Wang et al., J. Mater. Sci. Lett. 14 (1995), 490. The hydroxyapatite particles present there have a particle fineness of 2-10 nm and therefore belong to the range of the amorphous or partially X-ray amorphous substances. Hydroxyapatite nanoparticles are better suited which have a clearly discernible crystalline morphology, whose particle fineness is, therefore, in the range from 10-300 nm. Composite materials are likewise more suitable in which the finely divided poorly soluble calcium salts having particle finenesses of 10-300 nm form, together with finely divided proteins, protein hydrolyzates or derivatives thereof, a spatial structure in such a way that the finely divided calcium salts of the protein structure are aggregated and represent these quasi-spatially. Composite materials consisting of such preferably suitable nanoparticulate calcium salts and protein components lead to a particularly effective biomineralization.

Composite materials suitable according to the invention can be prepared by precipitation from aqueous solutions of water-soluble calcium salts with aqueous solutions of water-soluble phosphate and/or fluoride salts in the presence of protein components.

This is preferably carried out in such a way that the protein components are admixed in pure, dissolved or colloidal form to the alkaline aqueous phosphate and/or fluoride salt solution or to the alkaline solution of the calcium salt before the precipitation reaction. Alternatively, the protein components can be introduced in pure, dissolved or colloidal form and then treated successively in any desired sequence or simultaneously with the alkaline calcium salt solution, and also the alkaline phosphate and/or fluoride salt solution.

In the preparation process, the mixing together of the individual components can fundamentally take place in all possible sequences. The alkalizing agent used is preferably ammonia. In all precipitation reactions of this type, the pH of the precipitated system should be above pH=5.

A further variant of the preparation process consists in carrying out the precipitation from an acidic solution of a water-soluble calcium salt together with a stoichiometric amount of a water-soluble phosphate and/or fluoride salt or from an acidic solution of hydroxyapatite having a pH of below 5, preferably at a pH of below 3, by raising the pH using aqueous alkali or ammonia to a value of above 5 in the presence of the protein components.

A further process variant consists in treating nano-particulate calcium salts in pure or dispersed form or dispersions of nanoparticulate calcium salts prepared by precipitation reactions from aqueous solutions of water-soluble calcium salts and aqueous solutions of water-soluble phosphate and/or fluoride salts with the protein components, the latter preferably in dissolved or dispersed form, it being possible to choose any desired sequence during the addition.

Preferably, the solution or dispersion of the protein component is introduced and a dispersion of the nano-particulate calcium salt is added.

In all processes in the course of which a precipitation of apatite takes place, it is recommended to keep the pH above 5.

In all preparation processes mentioned, the resulting dispersion of the composite material can be separated off, if required, from the solvent, and the other constituents of the reaction mixture by processes known to the person skilled in the art, such as, for example, filtration or centrifugation, and isolated in solvent-free form by subsequent drying, e.g., by freeze-drying.

The solvent used in all preparation processes is preferably water, but in individual steps of the preparation organic solvents such as, for example, alcohols having 1 to 4 C atoms or glycerol can also be used.

In a particular embodiment of the invention, the finely divided calcium salt primary particles or the finely divided calcium salt primary particles present in the composite materials can be coated by one or more surface modification agents.

It is possible thereby, for example, to facilitate the preparation of composite materials in those cases in which the nanoparticulate calcium salts are difficult to disperse. The surface modification agent is adsorbed on the surface of the nanoparticle and modified in such a way that the dispersibility of the calcium salt increases and the agglomeration of the nanoparticle is prevented.

Moreover, the structure of the composite materials and the loading of the protein component with the nanoparticulate calcium salt can be influenced by surface modification. In this way, it is possible in the use of the composite materials in remineralization processes to bring an influence to bear on the course and the rate of the remineralization process.

Surface modification agents are to be understood as meaning substances which adhere physically to the surface of the finely divided particles, but do not react chemically with these. The individual molecules of the surface modification agents adsorbed on the surface are essentially free of intermolecular bonds with one another. Surface modification agents are, in particular, to be understood as meaning dispersants. Dispersants are known to the person skilled in the art under the terms “surfactants” and “protective colloids.” Suitable surfactants or polymeric protective colloids can be inferred from German Application No. DE 198 58 662 A1.

The composite materials according to the invention, in which the primary particles of the calcium salts are surface-modified, can be prepared by precipitation processes analogous to those described above, but where the precipitation of the nanoparticulate calcium salts or of the composite materials takes place in the presence of one or more surface modification agents.

Preferably, the surface-modified nanoparticulate calcium salts are firstly produced by a precipitation reaction between aqueous solutions of calcium salts and aqueous solutions of phosphate and/or fluoride salts in the presence of the surface modification agents. These can then be purified from accompanying products of the reaction mixture, e.g., by concentration under reduced pressure and subsequent dialysis. By stripping off the solvent, a dispersion of the surface-modified calcium salt with a solid component can additionally be prepared if desired. The composite material is then formed from surface-coated calcium salt and protein components by addition of the protein components in pure, dissolved or colloidal form, the sequence of the addition again not being critical, and, if necessary, subsequent reaction at elevated temperature, preferably in the range between 50 and 100° C. and for a period of 1 to 100 minutes.

For the preparation of the dental adhesive film according to the invention, the still liquid solution of the support material in water or aqueous alcohol is added to the active compound, that is the finely divided, poorly water-soluble calcium salt or preferably the composite material of the poorly soluble calcium salt and a protein component. For this, the active compound can be used as a water- and solvent-free powder or alternatively as an aqueous or aqueous-alcoholic dispersion. Finally, the dispersion obtained in this case is dried in a thin layer on a nonadhering substrate. The addition amount depends here on how much of the active compound is to be contained in the finished dental adhesive film. In a preferred embodiment of the invention, the active compound is contained in the ready-to-use dental adhesive film in an amount from 0.1-10% by weight.

Additionally to the remineralizing, finely divided, poorly water-soluble calcium salt contained according to the invention, further active compounds which are favorable for the health of the teeth or of the gums and are compatible with the support material can be contained. Such further active compounds are, for example
caries-inhibiting fluorine compounds, e.g., sodium fluoride, tin fluoride or sodium monofluoro-phosphate,
anti-tartar active compounds, e.g., organo-phosphates such as 1-hydroxyethane-1,1-di-phosphonic acid, phosphonopropane-1,2,3-tri-carboxylic acid (Na salts), 1-azacycloheptane-2,2-diphosphonic acid (Na salt),
desensitizing active compounds such as, for example, potassium nitrate or oil of cloves (eugenol),
wound-healing and anti-inflammatory substances such as, for example, allantoin, urea, azulene, camomile active compounds, thiocyanate,
deodorizing and antimicrobial substances such as, for example, chlorhexidine, hexetidine, bromo-chlorophene.

Further auxiliaries for improving the organoleptic properties can likewise be contained, e.g.
essential oils such as, for example, peppermint oil, spearmint oil, eucalyptus oil, aniseed oil, fennel oil, caraway oil, fruit aromas and synthetic essential oils,
sweeteners such as, for example, saccharin sodium, acesulfam-K, Aspartame®, sodium cyclamate, stevioside, thaumatin, sucrose, lactose, maltose, fructose or glycyrrhicin,
colorants and pigments.

The following Examples are intended to illustrate the subject of the invention in greater detail:

### EXAMPLES

Preparation of protein solutions or dispersions.

1.1 Gelatine type A.

10 g of gelatine type A (gelatine obtained by acidic hydrolysis of pigskin) were treated with 100 ml of water and firstly boiled by means of a microwave.

1.2 Gelatine type A and casein. 10 g of gelatine type A were treated with 100 ml of water and 10 ml of the supernatant of a casein solution saturated at 20° C. and then centrifuged at 5,000 rpm and then firstly boiled by means of a microwave.

1.3 Hydrolyzate of gelatine type A. 10 g of gelatine type A were treated with 100 ml of water and the alkaline protease Savinase (manufacturer: Novo Nordisk) in a use concentration of 0.005% enzyme dry matter, based on the dry matter of the gelatine. After stirring at 20° C. for 20 h, the mixture was firstly boiled by means of a microwave.

1.4 Hydrolyzate of gelatine type A and casein. 10 g of gelatine type A and 1 g of casein were treated with 100 ml of H₂O, hydrolyzed overnight at room temperature using alkaline protease Savinase (manufacturer: Novo Nordisk) in a use concentration of 0.005% enzyme dry matter, based on the dry matter of the protein components, then firstly boiled in the microwave and subsequently filtered.

1.5 Gelatine type B. 10 g of gelatine type B (gelatine obtained by alkaline hydrolysis of pigskin) were treated with 100 ml of water and firstly boiled by means of a microwave.

1.6 Gelatine type B and casein. 10 g of gelatine type B were treated with 100 ml of water and 10 ml of the supernatant of a casein solution saturated at 20° C. and then centrifuged at 5000 rpm and then firstly boiled by means of a microwave.

1.7 Hydrolyzate of gelatine type B. 10 g of gelatine type B were treated with 100 ml of water and the alkaline protease Savinase (manufacturer: Novo Nordisk) in a use concentration of 0.005% enzyme dry matter, based on the dry matter of the gelatine. After stirring at 20° C. for 20 h, the mixture was firstly boiled by means of a microwave.

1.8 Hydrolyzate of gelatine type B and casein. 10 g of gelatine type B and 1 g of casein were treated with 100 ml of H₂O, hydrolyzed overnight at room temperature using alkaline protease Savinase (manufacturer: Novo Nordisk) in a use concentration of 0.005% enzyme dry matter, based on the dry matter of the protein components, then first boiled in the microwave and subsequently filtered.

2. Preparation of composite materials by precipitation reactions in the presence of the protein components.

2.1 Composite material from hydroxyapatite and gelatine type A/ 2.21 g of calcium chloride were dissolved in 137 ml of completely demineralized water, temperature controlled at 25° C. and adjusted to pH=11 using 25% strength by weight aqueous ammonia solution. 20 ml of the protein solution prepared according to Example 1.1 heated in a water bath to 30-40° C. were then added with vigorous stirring. Following this, an aqueous solution of 1.58 g of diammonium hydrogenphosphate in 26 ml of completely demineralized water, which had been temperature controlled at 25° C. and adjusted to pH=11 using ammonia solution, was slowly added dropwise in the course of 1 h. In the course of this, the precipitation of the composite material took place. The pH at the start of the dropwise addition time was 10.4 and was kept at a value of about 10 by subsequent addition of ammonia solution. After a reaction time of 20 h (25° C., with stirring), the pH of the aqueous suspension had fallen to 9.5. The precipitated composite material was centrifuged off at 5,000 rpm, washed with completely demineralized water at about 30-40° C. and freeze-dried. 2.2 g of composite material were obtained, whose elemental analysis showed a carbon content of 2.3%; this corresponds to a content of protein material of 5.6% by weight, based on the total amount of the composite material.

2.2-2.8 Composite materials of hydroxyapatite and further protein components.

In a manner analogous to that described in Example 2.1, composite materials were obtained from hydroxyapatite and the protein components described in 1.2 to 1.8.

3. Preparation of composite materials by incorporation of dispersions of surface-modified calcium salts into protein components.

3.1 Composite material from hydroxyapatite and gelatine Bloom 300:

The solutions A and B were firstly prepared separately. Solution A: 25.4 g of calcium nitrate tetrahydrate and 8.50 g of diammonium hydrogenphosphate were in each case dissolved in 100 ml of deionized water. Both solutions were mixed together with the formation of a white precipitate. After addition of 10 ml of 37% strength by weight HCI, a clear solution was obtained.

Solution B: 200 ml of deionized water, 200 ml of 25% strength by weight aqueous ammonia solution and 20 g of Plantacare®1200 were mixed together and cooled to 0° C. in an ice bath.

Solution A was added to solution B with vigorous stirring with formation of a hydroxyapatite precipitate. After stripping off excess ammonia, the dispersion was purified by means of dialysis. The dispersion was then concentrated on a rotary evaporator by determination of the amount of water separated until the solids content in the dispersion, calculated as hydroxyapatite, was 7.5% by weight.

This dispersion was added at room temperature to 100 ml g of a 10% strength by weight aqueous solution of gelatine Bloom 300 (manufacturer: Fluka) prepared analogously to Example 1.1, then heated to 80° C. and stirred at this temperature for 5 minutes. The mass was then allowed to solidify with formation of the composite material at room temperature.

4. Preparation of dental adhesive films.

4.1 PVAc/HPC film

A dispersion of the composite material in aqueous alcoholic solution of polyvinyl acetate and hydroxy-propylcellulose of the following composition was prepared.

| | |
|---|---|
| Polyvinyl acetate (M.W. 172,000) | 5% by weight |
| Hydroxypropylcellulose | 5% by weight |
| Water | 9% by weight |
| Methanol | 80% by weight |
| Composite material | 1% by weight |

The dispersion was poured in a layer 2 m thick onto a silicone-coated substrate and dried. A film about 0.2 mm thick was obtained, which was cut into tapes 1 cm wide.

4.2 Gelatine film.

| | |
|---|---|
| Gelatine hydrolyzate | 10.0% by weight |
| Composite material according to | 1.0% by weight |
| Example 3.1 | |
| Ethanol | 45.0% by weight |
| Water | 35.0% by weight |
| Galloylgallic acid | 9.0% by weight |

The dispersion was poured in a layer 2 mm thick onto a silicone-coated substrate and dried. A film about 0.2 mm thick was obtained, which was cut into tapes about 1 cm wide.

1. A dental adhesive film for local, remineralizing tooth treatment comprising a water soluble or swellable support material for adhering to the tooth comprising a composite comprising at least one active remineralizing compound incorporated into the support material wherein the active compound is a poorly water-soluble calcium salt of a compound selected from the group consisting of fluorides, fluorophosphates and mixtures thereof in the form of a finely divided rod-shaped nanoparticle having a mean particle fineness of from 10 to 300 nm and a protein component selected from the group consisting of proteins, and protein degradation products and derivatives of proteins or protein degradation products wherein the composite is an aggregate which is microscopically heterogeneous but macroscopically homogeneous.

2. The dental adhesive film of claim 1, further comprising hydroxyl, carbonate or chloride ions.

3. The dental adhesive film of claim 1, wherein the finely divided calcium salt is fluoroapatite.

4. The dental adhesive film of claim 1, wherein the protein component is selected from the group consisting of gelatine, casein, their hydrolyzates and mixtures thereof.

5. The dental adhesive film of claim 1, wherein the protein component is contained in an amount of between 0.1% and 60% by weight, based on the weight of the composite material.

6. The dental adhesive film of claim 1, wherein the support material is a water-soluble or water-swellable, natural or synthetic polymer material, selected from the group consisting of plant and microbial gums, cellulose ethers, copolymers of acrylic or methacrylic acid and esters of acrylic or methacrylic acid, polyvinyl alcohol, partially hydrolyzed polyvinyl acetate, polyvinylpyrrolidone and mixtures thereof.
